(19) United States
(12) Patent Application Publication
(10) Pub. No.: US 2011/0208050 A1
(43) Pub. Date: Aug. 25, 2011

(54) ULTRASONIC DIAGNOSTIC APPARATUS

(75) Inventor: Suk Won KIM, Gyeonggi-do (KR)

(73) Assignee: MEDISON CO., LTD.

(21) Appl. No.: 12/971,728

(22) Filed: Dec. 17, 2010

(30) Foreign Application Priority Data
Feb. 24, 2010 (KR) 10-2010-0016820

(51) Int. Cl. A61B 8/00

(52) U.S. Cl. 600/437

(57) ABSTRACT

An ultrasonic diagnostic apparatus is disclosed. The ultrasonic diagnostic apparatus includes a display unit displaying an ultrasound image, and an object checking unit checking a state or probing portion of an object.

According to the ultrasonic diagnostic apparatus, the display unit for displaying an ultrasound image and the object checking unit for checking a state or probing portion of an object are located in the same direction, so that an examiner views the ultrasound image and the state of the object without alternately turning the head to view the monitor and the object.

### BACKGROUND OF THE INVENTION

1. Field of the Invention

The present invention relates to an ultrasonic diagnostic apparatus and, more particularly, to an ultrasonic diagnostic apparatus that may monitor an ultrasound image of an object and a state or probing portion of the object in one direction.

2. Description of the Related Art

Generally, an ultrasonic diagnostic apparatus refers to a non-invasive apparatus that irradiates an ultrasound signal from a surface of a patient body towards a target internal organ beneath the body surface and obtains an image of a monolayer or blood flow in soft tissue from information in the reflected ultrasound signal (ultrasound echo-signal).

The ultrasonic diagnostic apparatus has been widely used for diagnosis of the heart, the abdomen, the urinary organs, and in obstetrics and gynecology due to various merits such as small size, low price, real-time image display, and high stability through elimination of radiation exposure, as compared with other image diagnostic apparatus, such as X-ray diagnostic apparatus, computerized tomography scanners (CT scanners), magnetic resonance imagers (MRIs), nuclear medicine diagnostic apparatuses, and the like.

The ultrasonic diagnostic apparatus includes a probe that transmits an ultrasound signal to an object and receives the ultrasound echo-signal reflected therefrom to obtain an ultrasound image of the object.

The signal received by the probe is output to a monitor of the apparatus through a controller and an operator examines the object while alternately viewing the monitor and the object.

Here, it should be noted that the above description is provided for understanding of the background of the invention and is not a description of a well-known conventional technique in the art to which the present invention pertains.

As such, since the examiner examines the object while alternately viewing the monitor and the object on which the probe is positioned, the examiner is likely to become tired and suffers low operation efficiency. Therefore, there is a need for an improved ultrasonic diagnostic apparatus that overcomes such a problem.

### SUMMARY OF THE INVENTION

The present invention is conceived to solve the problem of the related art, and an aspect of the invention is to provide an ultrasonic diagnostic apparatus that allows an examiner to view an ultrasound image and a state of an object without alternately turning the head to view a monitor and the object.

In accordance with an aspect of the invention, an ultrasonic diagnostic apparatus includes: a display unit displaying an ultrasound image; and an object checking unit checking a state or probing portion of an object.

The object checking unit may include a photographing unit capturing an image of the object and an object display unit displaying the image of the object captured by the photographing unit.

The object display unit may be separated from the display unit.

The object display unit may be located at a lateral side of the display unit.

The object display unit may be the display unit.

The object checking unit may include a mirror at a lateral side of the display unit.

The mirror may include a convex mirror.

### BRIEF DESCRIPTION OF THE DRAWINGS

The above and other aspects, features and advantages of the invention will become apparent from the following description of embodiments given in conjunction with the accompanying drawings, in which:

FIG. 1 is a schematic perspective view of an ultrasonic diagnostic apparatus according to one embodiment of the present invention;

FIG. 2 is a schematic perspective view of an ultrasonic diagnostic apparatus according to another embodiment of the present invention; and

FIG. 3 is a schematic perspective view of an ultrasonic diagnostic apparatus according to a further embodiment of the present invention.

### DETAILED DESCRIPTION OF THE EMBODIMENT

Exemplary embodiments of the invention will now be described in detail with reference to the accompanying drawings. For convenience of description, an ultrasonic diagnostic apparatus including a probe will be described herein. It should be noted that the drawings are not to precise scale and may be exaggerated in thickness of lines or size of components for descriptive convenience and clarity only. Furthermore, terms used herein are defined by taking functions of the invention into account and can be changed according to the custom or intention of users or operators. Therefore, definition of the terms should be made according to overall disclosures set forth herein.

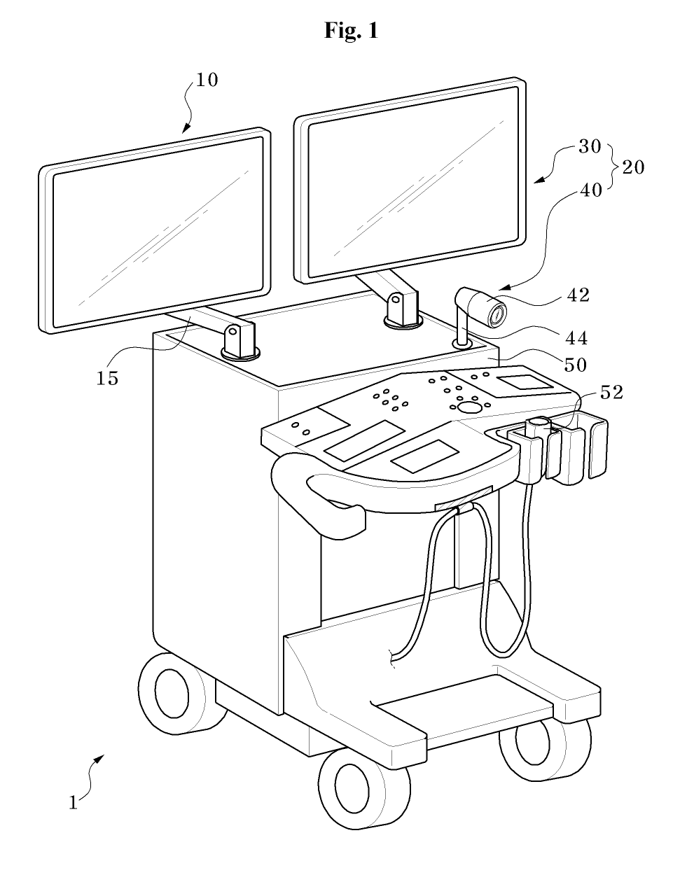

FIG. 1 is a schematic perspective view of an ultrasonic diagnostic apparatus according to one embodiment of the present invention.

Referring to FIG. 1, the ultrasonic diagnostic apparatus 1 according to the embodiment includes a display unit 10 that displays an ultrasound image, and an object checking unit 20 that checks a state or probing portion of the object.

As for the display unit 10, any kind of imaging apparatus such as a CRT (cathode ray tube) imaging apparatus or an LCD (liquid crystal display) imaging apparatus may be used so long as the imaging apparatus can display an ultrasound image.

The display unit 10 is supported by a support member 15 and may be rotated or moved to a desired location, as needed.

The object checking unit 20 for checking a state or probing portion of the object includes a photographing unit 40 that captures an image of the object, and an object display unit 30 that displays the image of the object captured by the photographing unit 40.

As for the photographing unit 40, any kind of imaging apparatus may be used so long as the imaging apparatus can capture an image of an object. In one embodiment, the photographing unit 40 includes a body member 42 that receives an image from an outside, and a connector 44 that connects the body member 42 to a main body 50 of the diagnostic apparatus.

The photographing unit 40 may be automatically or manually moved according to movement of an object or probing portion of the object.

When the photographing unit 40 is configured to move automatically, a probe 52 is provided with a signal transmitter for position recognition, the main body 50 is provided with a signal receiver for receiving a signal sent from the signal transmitter, and the photographing unit 40 is rotated towards the probe 52.

Alternatively, the photographing unit 40 may be rotated by operating a separate remote controller or an operation button to photograph a state or probing portion of an object. Obviously, other various modifications of the photographing unit may also be embodied.

When the photographing unit 40 is configured to be moved manually, a position of the photographing unit 40 is adjusted by directly rotating the body member 42 or the connector 44 by an operator to confirm the state or probing portion of the object.

As for the object display unit 30, any kind of imaging apparatus such as a CRT imaging apparatus or an LCD imaging apparatus may be used so long as the imaging apparatus can output an image captured by the photographing unit 40.

The object display unit 30 is also supported by the support member 15 and may be rotated or moved to a desired location, as needed.

The object display unit 30 is separated from the display unit 10. The object display unit 30 may be located at a lateral side of the display unit 10, so that the state or probing portion of the object and an ultrasound image of the object may be confirmed at the same time in one direction by an operator.

The display unit 10 and the object display unit 30 may be provided to the main body 50 of the diagnostic apparatus 50, to which the probe 52 is connected to send a scanned image to the main body 50.

Next, operation of the ultrasonic diagnostic apparatus according to the embodiment will be described with reference to the drawings.

An ultrasound image of an object scanned by the probe 52 is output on the display unit 10 and an image of a state or probing portion of the object captured by the photographing unit 40 is output on the object display unit 30.

Since the display unit 10 and the object display unit 30 are parallel to each other while being separated from each other, an examiner may perform examination of the object on the display unit 10 and object display unit 30 without separately viewing the object, so that operation efficiency is improved.

In the ultrasonic diagnostic apparatus 1 of the embodiment described above, the display unit 10 for outputting an ultrasound image of an object and the object display unit 20 for checking a state or probing portion of the object are disposed in the same direction to allow an examiner to check the ultrasound image and the state of the object at the same time without turning the head, so that the examiner experiences less fatigue and can perform examination of the object with high operation efficiency.

Next, an ultrasonic diagnostic apparatus according to another embodiment will be described.

For convenience of description, the same elements as those of the above embodiment will be indicated by the same reference numerals and a detailed description thereof will be omitted herein.

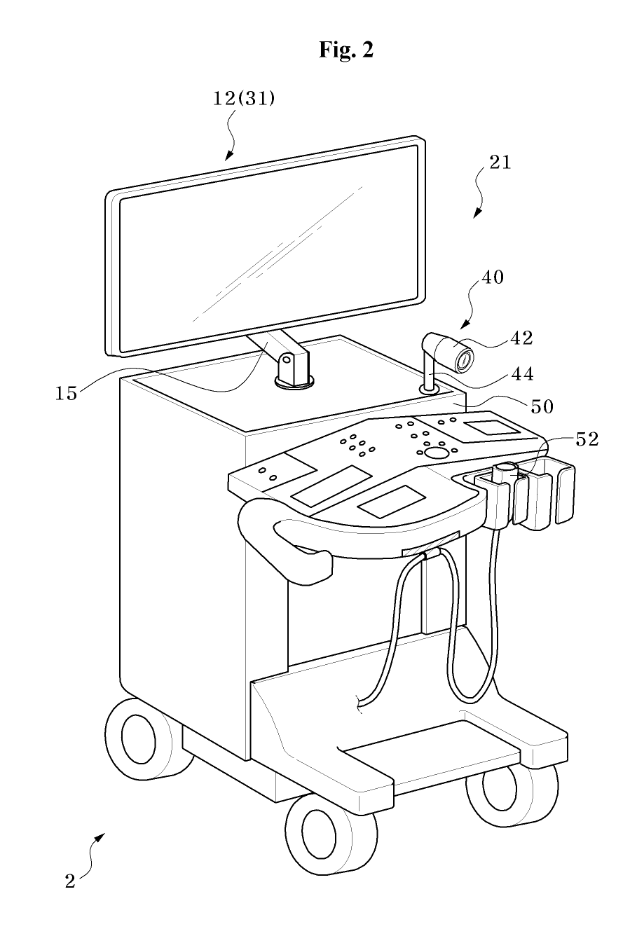

FIG. 2 is a schematic perspective view of an ultrasonic diagnostic apparatus according to another embodiment of the present invention.

Referring to FIG. 2, an ultrasonic diagnostic apparatus 2 of this embodiment includes a display unit 12 and an object checking unit 31, which are integrally formed with each other.

In this embodiment, the display unit 12 has a multi-divisional screen and allows an ultrasound image of an object sent from a probe 52 and an image relating to a state or probing portion of the object sent from a photographing unit 40 to be displayed on divided screens of the display unit 12.

In this embodiment, the display unit 12 uses a wide screen, which permits easy division of the screen.

Next, an ultrasonic diagnostic apparatus according to a further embodiment will be described.

For convenience of description, the same elements as those of the above embodiments will be indicated by the same reference numerals and a detailed description thereof will be omitted herein.

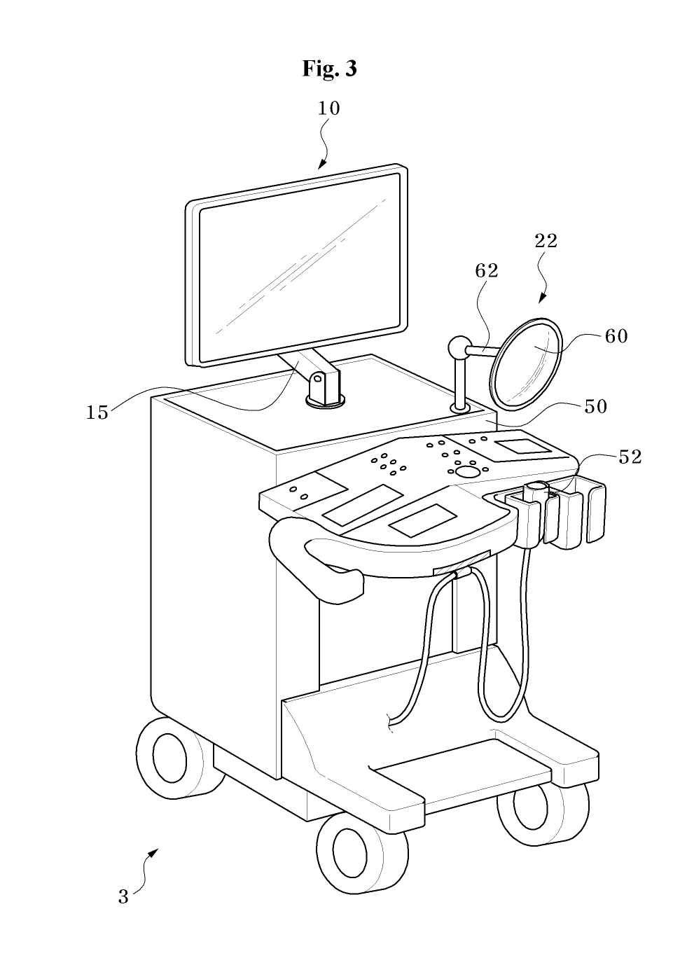

FIG. 3 is a schematic perspective view of an ultrasonic diagnostic apparatus according to a further embodiment of the present invention.

Referring to FIG. 3, an ultrasonic diagnostic apparatus 3 of this embodiment includes an object checking unit 22, which includes a mirror 60 at a lateral side of the display unit 10.

The mirror 60 is supported by a mirror support 62 and is connected to the main body 50 of the diagnostic apparatus. The mirror 60 is manually or automatically rotatable.

A configuration of allowing automatic rotation of the object checking unit 22 is apparent to those skilled in the art, and a detailed description thereof will be omitted herein.

As for the mirror 60, any kind of mirror may be used so long as it can be used to check a state or probing portion of an object. In another embodiment, the object checking unit 22 includes a convex mirror to secure a wide viewing angle.

As such, in the ultrasonic diagnostic apparatus according to the embodiments, a display unit for displaying an ultrasound image and an object checking unit for checking a state or probing portion of an object are located in the same direction, so that an examiner views the ultrasound image and the state of the object without alternately turning the head to view the monitor and the object. Thus, the ultrasonic diagnostic apparatus of the embodiments may improve operation efficiency of the examiner by lowering fatigue of the examiner.

Although some embodiments have been provided to illustrate the invention in conjunction with the drawings, it will be apparent to those skilled in the art that the embodiments are given by way of illustration only, and that various modifications and equivalent embodiments can be made without departing from the spirit and scope of the invention.

Further, although the ultrasonic diagnostic apparatus using the probe is provided in description of the embodiments, it is obvious that it is given by way of illustration only and the present invention may also be applied to various kinds of diagnostic apparatus.

Therefore, the scope of the invention should be limited only by the accompanying claims and equivalents thereof.

What is claimed is:

1. An ultrasonic diagnostic apparatus comprising:
a display unit displaying an ultrasound image; and
an object checking unit checking a state or probing portion of an object.

2. The ultrasonic diagnostic apparatus of claim 1, wherein the object checking unit comprises:
a photographing unit capturing an image of the object; and
an object display unit displaying the image of the object captured by the photographing unit.

3. The ultrasonic diagnostic apparatus of claim 2, wherein the object display unit is separated from the display unit.

4. The ultrasonic diagnostic apparatus of claim 3, wherein the object display unit is located at a lateral side of the display unit.

5. The ultrasonic diagnostic apparatus of claim 2, wherein the object display unit is the display unit.

6. The ultrasonic diagnostic apparatus of claim 1, wherein the object checking unit comprises a mirror at a lateral side of the display unit.

7. The ultrasonic diagnostic apparatus of claim 6, wherein the mirror is a convex mirror.
